(19) United States
(12) Patent Application Publication
(10) Pub. No.: US 2009/0157064 A1
(43) Pub. Date: Jun. 18, 2009

(54) RFID System and Method Therefor

(76) Inventor: Michael R. Hodel, Fremont, CA (US)

Correspondence Address: AMS RESEARCH CORPORATION, 10700 BREN ROAD WEST, MINNETONKA, MN, 55343, US

(21) Appl. No.: 12/119,298

(22) Filed: May 12, 2008

Related U.S. Application Data
(60) Provisional application No. 60/917,485, filed on May 11, 2007.

(51) Int. Cl. A61B 18/22; G06K 7/06

(52) U.S. Cl. 606/10; 235/441

(57) ABSTRACT

A system and method for authenticating an optical fiber for use with a medical laser system includes a medical laser unit, an optical fiber, and a memory device. The laser unit is typically specific to a certain medical procedure and is under the operation of a control system. The optical fiber is operably coupled to the laser unit and includes a probe tip for position proximate laser-targeted tissue. The memory device is associated with the optical fiber, e.g., as an RFID tag embedded in fiber or a smart card operational with the fiber, and is configured to allow the control system of the laser unit to access information embedded in the memory device so that the optical fiber may be authenticated as suitable to operate with the laser unit.

### PRIORITY CLAIM

The present application claims priority to U.S. Provisional Application Ser. No. 60/917,485 filed May 11, 2007, and entitled “RFID SYSTEM AND METHOD THEREFOR”, which is hereby incorporated by reference in its entirety.

### FIELD OF THE DISCLOSURE

This invention relates to the field of medical lasers utilizing optical fibers. More specifically, the present invention relates to the use of radio frequency identification tags to authenticate an optical fiber.

### BACKGROUND OF THE INVENTION

Medical lasers have been used in treatment procedures involving various practice areas, including, for example, urology, neurology, otorhinolaryngology, general anesthetic ophthalmology, dentistry, gastroenterology, cardiology, gynecology, and thoracic and orthopedic procedures. Generally, these procedures require precisely controlled delivery of laser energy, and often the area to which the laser energy is to be delivered is located deep within the body; for example, at the prostate or at the fallopian tubes. Due to the location of the target tissue deep within the body, the medical procedure requires that the optical fiber be flexible and maneuverable. Various light sources can be used with optical fiber devices dependent upon the requirements for the light source; for example, pulsed lasers, diode lasers and neodymium lasers can be used as light sources. Representative lasers used in medical treatment procedures include Ho:YAG lasers and Nd:YAG lasers.

In medical procedures utilizing laser energy, the laser is coupled to an optical fiber adapted to direct laser radiation from the laser, through the fiber and to the treatment area. Typically, a surgical probe is utilized in the treatment of body tissue with laser energy. The surgical probe generally includes an optical fiber coupled to a laser source, and the probe tip is positioned on the optical fiber opposite the laser source, such that the tip of the probe can be positioned adjacent to the targeted tissue. Laser energy is directed out of the probe tip of the optical fiber onto desired portions of the targeted tissue. There are many varieties of medical optical fibers available in the marketplace that can be used with laser systems that are used in medical procedures. These laser systems provide laser light at various wavelengths, and are used for various particular purposes and procedures. The optical fibers used with these laser systems can be made of various materials, operate at various temperatures, operate at various wavelengths, and have various bend radii. There are a variety of materials that can be used for the core of the optical fiber, for the cladding, for the buffer, and for the jacket, resulting in many combinations. Prior to beginning a medical procedure, it is important that the proper optical fiber be connected to the laser unit that is to be used for the medical procedure. Oftentimes the manufacturer of the laser unit recommends usage of particular brands of optical fibers and/or particular optical fibers with the laser unit. Use of an improper optical fiber can result in damage to the equipment, delay in conducting a medical procedure until the proper optical fiber is obtained, and a potential for an ineffective or even damaging medical procedure. Thus, there remains an ongoing need for a method or mechanism for reliable and convenient monitoring of optical fibers.

### SUMMARY OF THE INVENTION

The present invention generally comprises a system and/or method for authenticating an optical fiber for use with a medical laser system. In such a system, the elements generally include a medical laser unit, an optical fiber, and a memory device. The laser unit is typically specific to a certain medical procedure and is under the operation of a control system. The optical fiber is operably coupled to the laser unit and includes a probe tip for position proximate laser-targeted tissue. The memory device is associated with the optical fiber, e.g., as an RFID tag embedded in fiber or a smart card operational with the fiber, and is configured to allow the control system of the laser unit to access information embedded in the memory device so that the optical fiber may be authenticated as suitable to operate with the laser unit.

In a preferred system embodiment, the control system and the memory device share a common authentication key. The authentication key allows the memory device to generate a unique answer in response to a non-repeated prompt from the control system. Additionally, the memory device preferably includes a settable memory region whereby the manufacturer may set identity number that are proprietary to the supplier of the laser unit. The memory device may be further used to communicate operational history information of the optical fiber to the control system. In a preferred embodiment, it is further possible to utilize the control system to deauthenticate the memory device of the optical fiber to prevent further use of the fiber within the laser unit. In a preferred embodiment, the laser unit is specifically configured to provide photoselective vaporization of prostate tissue through the optical fiber.

A method of authenticating an optical fiber for use with a medical laser includes the steps of: providing an optical fiber having a memory device specific to the optical fiber, the memory device including fiber information unique to the optical fiber; connecting the fiber to a laser unit; and authenticating use of the optical fiber with the laser unit by accessing the fiber information with a control system on the laser unit, whereby the control system verifies compatibility of the optical fiber with the laser unit.

### BRIEF DESCRIPTION OF THE DRAWINGS

These as well as other objects and advantages of this invention, will be more completely understood and appreciated by referring to the following more detailed description of the presently preferred exemplary embodiments of the invention in conjunction with the accompanying drawings of which:

FIG. 1 is a block diagram illustration of a representative KTP laser.

FIG. 2 is a plan view of an optical fiber including an RFID device according to an embodiment of the present invention.

FIG. 3 is a front, perspective view of a medical laser unit according to an embodiment of the present invention.

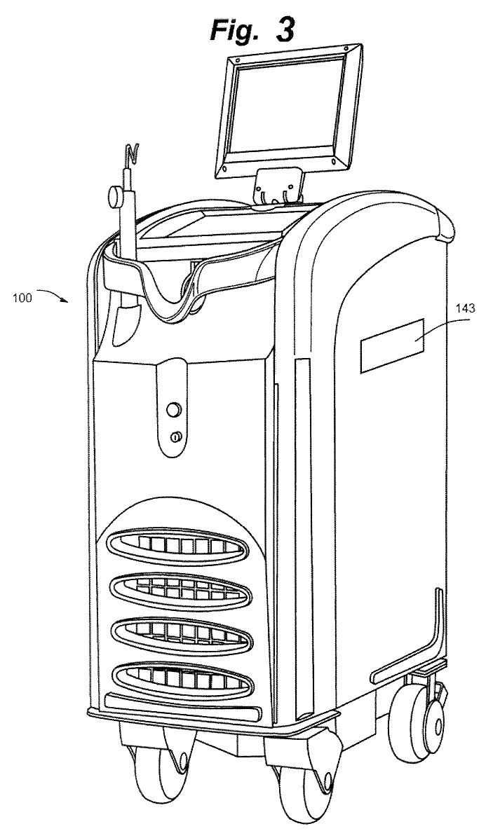

FIG. 4 is a flow chart illustrating a method of authenticating an optical fiber according to an embodiment of the present invention.

FIG. 5 is a flow chart illustrating a method of authenticating an optical fiber according to an embodiment of the present invention.

While the invention is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives.

### DETAILED DESCRIPTION OF THE PREFERRED EMBODIMENTS OF THE INVENTION

The present invention includes an authentication scheme for fibers utilized with medical lasers. Specifically, the present invention directed to authentication of fibers in the Greenlight HPS laser treatment product, manufactured and sold by American Medical Systems, Inc. The Greenlight PVP and HPS comprise laser treatments for soft tissue and are particularly suited to photoselective vaporization of the prostate. The system and its operation are generally described in U.S. Pat. Nos. 6,554,824 and 6,986,764, which are hereby incorporated by reference. The authentication scheme comprises embedding a radio frequency identification (RFID) chip into the fiber connector to authenticate the fiber. Additional security and functionality can be obtained by also using a separate smart card to store data, validate service personnel, and obtain procedure information.

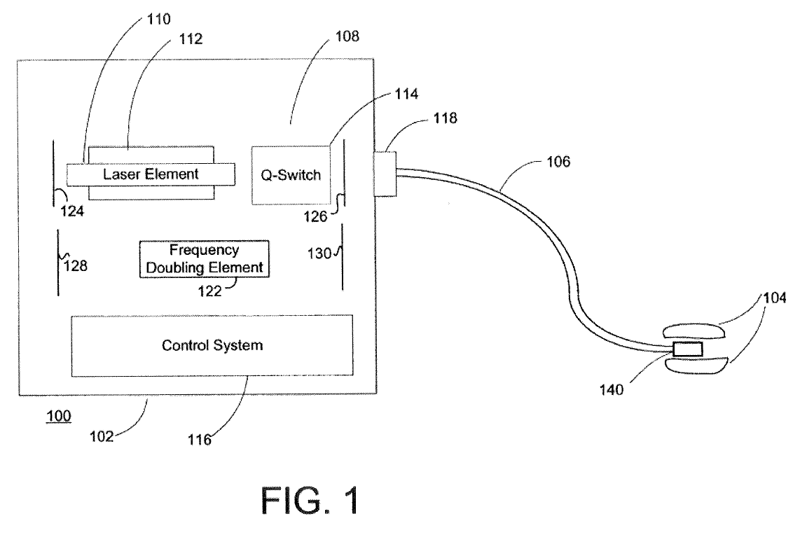

Referring to FIG. 1, there is depicted a block diagram showing an exemplary laser system 100 which may be employed for implementing the present invention. Laser system 100 includes a solid-state laser 102, which is used to generate laser light for delivery through optical fiber 106 to target tissue 104. Laser 102 is capable of being operated in a pulsed mode or continuous wave.

Laser 102 more specifically comprises a laser element assembly 110, pump source 112, and frequency doubling crystal 122. In the preferred-embodiment, laser element 110 outputs 1064 nm light which is focused into frequency doubling crystal 122 to create 532 nm light. According to one implementation, laser element assembly 110 may be neodymium doped YAG (Nd:YAG)crystal, which emits light having a wavelength of 1064 nm (infrared light) when excited by pump source 112. Laser element 110 may alternatively be fabricated from any suitable material wherein transition and lanthanide metal ions are disposed within a crystalline host (such as YAG, Lithium Yttrium Fluoride, Sapphire, Alexandrite, Spinel, Yttrium Orthoaluminate, Potassium Gadolinium Tungstate, Yttrium Orthovandate, or Lanthahum Scandium Borate). Laser element 110 is positioned proximal to pump source 112 and may be arranged in parallel relation therewith, although other geometries and configurations may be employed.

Pump source 112 may be any device or apparatus operable to excite laser element assembly 110. Non-limiting examples of devices which may be used as pump source 112, include: arc lamps, flashlamps, and laser diodes.

A Q-switch 114 disposed within laser 102 may be operated in a repetitive mode to cause a train of micropulses to be generated by laser 102. Typically the micropulses are less than 1 microsecond in duration separated by about 40 microseconds, creating a quasi-continuous wave train. Q-switch 114 is preferably of the acousto-optic type, but may alternatively comprise a mechanical device such as a rotating prism or aperture, an electro-optical device, or a saturable absorber.

Laser 102 is provided with a control system 116 for controlling and operating laser 102. Control system 116 will typically include a control processor which receives input from user controls (including but not limited to a beam on/off control, a beam power control, and a pulse duration control) and processes the input to accordingly generate output signals for adjusting characteristics of the output beam to match the user inputted values or conditions. With respect to pulse duration adjustment, control system 116 applies an output signal to a power supply (not shown) driving pump source 112 which modulates the energy supplied thereto, in turn controlling the pulse duration of the output beam.

Although FIG. 1 shows an internal frequency doubled laser, it is only by way of example. The infrared light can be internally or externally frequency doubled using non-linear crystals such as KTP, Lithium Triborate (LBO), or Beta Barium Borate (BBO) to produce 532 nm light. The frequency doubled, shorter wavelength light is better absorbed by the hemoglobin and char tissue, and promotes more efficient tissue ablation. Finally, the green light leaves only a thin char layer with little pre and post operative bleeding.

Laser 102 further includes an output port 118 couplable to optical fiber 106. Output port 118 directs the light generated by laser 102 into optical fiber 106 for delivery to tissue 104. While a bare fiber may be utilized for certain procedures, optical fiber 106 preferably terminates in a tip 140 having optical elements for shaping and/or orienting the beam emitted by optical fiber 106 so as to optimize the tissue ablation process, for example a side-firing fiber.

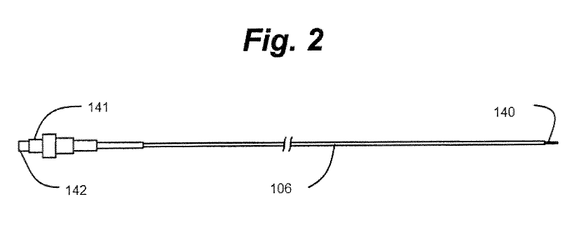

Referring to FIG. 2, there is depicted optical fiber 106 with tip 140 configured for connection with optical port 118 through use of a connector 141. In this preferred embodiment, optical fiber 106 additionally incorporates an RFID (radio frequency identification) device 142. RFID devices 142 are low power silicon devices that can be powered and communicate via an RF field. In this way the laser system 100 can generate a low power localized RF field that powers the RFID device (a.k.a. tag). Modulating this RF field also allows communication. This enables wireless communication to an embedded chip within optical fiber 106 that does not require its own power source.

RFID devices are effectively a non-volatile memory element. They contain an EEPROM (typically 320 to 4096 bytes) and interface logic that handles communications. RFID devices are compact, e.g., they can be fabricated in a small disk that can be embedded into connector 141. Of course other formats/manners of manufacturing RFID devices can be used without departing from the spirit or scope of the invention.

There are several security aspects to RFID devices 142 that lend themselves to their suitablility for fiber authentication within laser system 100:
1. Uniquely Identify a Laser Fiber. The RFID device 142 can be configured to uniquely identify that a fiber was made by a desired manufacturer, e.g., American Medical Systems, Inc., and prevent simple duplication of fiber information to create copy fibers. There are two ways to do this:
   a. Encrypted Authentication Codes The first and preferred approach to uniquely identifying a laser fiber is to authenticate the fiber by using a private key. In this approach both the laser system 100 and the RFID device 142 of the optical fiber 106 are taught an identical key. The RFID device 142 and laser system 100 then operate in conjunction to authenticate the key. Specifically, the laser system 100 generates a random, unique challenge number. The RFID device 142 uses this challenge, in combination with the key to generate a response of an authentication code. The method for generating this code (known as a hash function) masks the value of the key. So effectively, what is transmitted is the answer to a random, never to be repeated, question. This protects the value of the key and prevents duplication.
   b. Unique and Unchangeable Identity Numbers. This is the second and alternative approach to uniquely identifying a laser fiber. This approach can be used if there is a region of memory (e.g., a serial or model number), that can only be written by the RFID manufacturer. The protection is realized by ensuring that the manufacturer only provides tags with legal identification numbers, e.g., to the Greenlight HPS manufacturer. This prevents simple duplication of legitimate tags.
2. Deauthentication of Fibers. The next important security aspect of RFID devices 142 in conjunction with laser system 100 is that the RFID device can be configured such that fibers are able to be deauthenticated. So, once a fiber is used, it must indicate that it is no longer usable. This is accomplished on most any fiber by simply setting a memory location within the RFID device to a “no-longer-usable” code and then locking this memory location so that it can never be written to again. This feature is commonly supported by nearly all RFID devices.

There are several RFID devices that can be used to address the above-described needs. Some examples include, but are not limited to: 1. Philips Mifare DESfire—This is the preferred embodiment. It supports encrypted authentication and has sufficient memory for data storage; 2. Philips Hitag S—Another Philips tag that has encryption, however, it is not as secure and does not have as much memory as example 1; 3. ATMEL TK5561—This tag is similar to the Hitag S. It too has only a small amount of memory and less than ideal security; 4. Texas Instruments DST—This tag is also similar to the Hitag S.

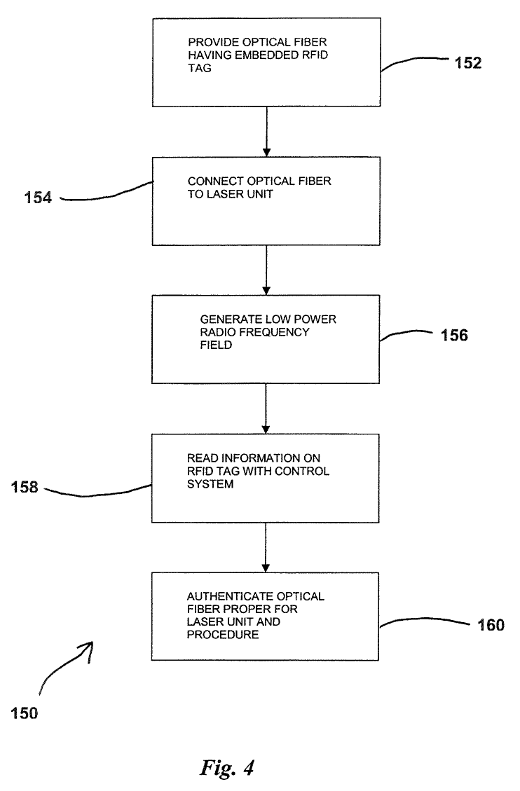

In another aspect of the present invention, the optical fiber authentication techniques can be provided by way of a smart card 143 system, instead of an RFID device 142. As illustrated in FIG. 4, the laser system 100 can include a smart card system 143. A smart card has much more memory available as compared to the RFID devices 142 noted above. The smart card provides secure authentication and many different card options exist, and different card types can be used in the same card reader. If information about the optical fiber 106 is to be transmitted back, the information can be sent via the smart card rather than the return of a used fiber. Further, the smart card can be used for service technician verification, which is preferable to using a RFID device 142 for this task. To provide additional security to either the RFID device system or the smart card system 143, both the smart card and the RFID device 142 can be used together, to provide additional security by requiring that two separate barriers be breached to implement copy optical fibers, or to use an optical fiber 106 that should not be used.

In use, an optical fiber 106 including an integral, embedded RFID device 142 can be connected and authenticated to the laser unit 100 using an authentication method 150 as illustrated in FIG. 4. Generally, optical fiber 106 including the RFID device 142 can be provided in a first step 152 and operably connected to the laser unit 100 at step 154. Once the optical fiber 106 is operably coupled to the laser unit 100, a low power radio frequency field can be generated at step 156 such that information embedded within RFID device 142 can be accessed and read at step 158 by the control system 116 in the laser unit 100. Utilizing the information ready at step 158, the control system 116 can authenticate the optical fiber 106 at step 160. At a minimum, step 160 involves confirming that optical fiber 106 is suitable for use with the laser unit 100. Additionally, it can be used to confirm that the optical fiber 106 is suitable for a specific laser treatment. Step 160 can include the use of encrypted authentication codes or by unique, manufacturer supplied identity numbers. For instance, the control system 116 and RFID device 142 can share a common authentication key by which non-repeated requests from the control system 116 can be responded to with unique, correct answers. Such an authentication method commonly referred to as a hash function in which the value of the shared key is always masked to protect the value of the key and prevent unauthorized duplication of the shared key.

Alternatively, RFID device 142 can include a factory settable memory region for storing manufacturer settable identity numbers, for example, model or serial number information, that are proprietary to the manufacturer of laser unit 100 and that must be confirmed in order to authenticate the optical fiber 106. As such, step 160 can assures that the proper optical fiber 106 is being used with the laser unit 100 for the proper medical procedure. Once authenticated, the medical procedure for the patient can be performed. After the medical procedure is performed, the optical fiber 106 can optionally be de-authenticated by the control system to prevent re-use or in the event that the optical fiber 106 is to be re-used following sterilization and inspection, the optical fiber 106 can be de-authenticated, and upon completion of the sterilization and inspection procedures, the optical fiber 106 can be re-authenticated for use.

In addition to authenticating the optical fiber 106, the method can further include accessing an operational history of the optical fiber 106. Operational history information for the optical fiber 106 can include, for example, run data that can include total laser power delivered, number of procedures performed and the like. Following completion of the medical procedure, the control system can transmit new operational data to the RFID device 142 so as to update the operational history of the optical fiber 106.

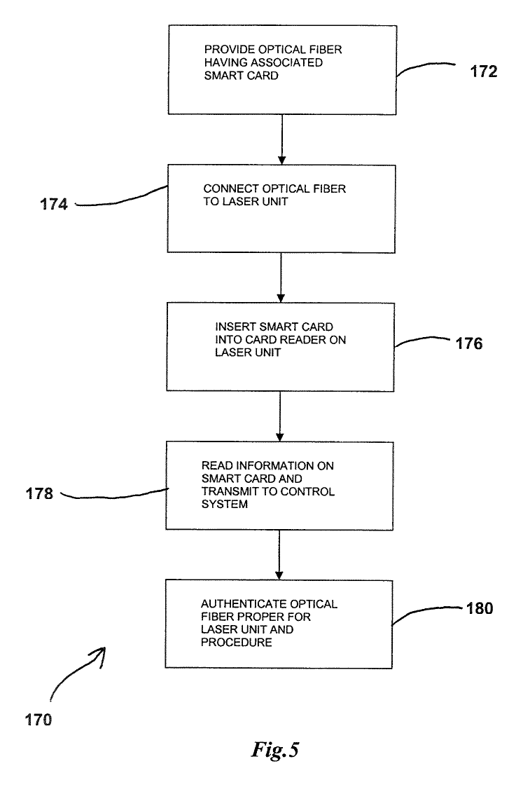

An alternative authentication method 170 utilizing a separate smart card system 143 is illustrated in FIG. 5. Generally, optical fiber 106 along with a corresponding smart card is provided in a first step 172. The optical fiber 106 is operably connected to the laser unit 100 at step 174 while the smart card is inserted into the laser unit 120 at step 176. Laser unit 120 can include a smart card reader for receiving the smart card, reading the information embedded on the smart card at step 178 and transmitting the information to the control system. Using the information obtained in step 178, the control system on the laser unit 100 can authenticate the optical fiber 106 based on the information contained on smart card at step 180. Once again, authentication involves at a minimum, confirming that optical fiber 106 is suitable for use with the laser unit 100. Authentication can additionally include determining if the optical fiber 106 is suitable for a specific laser treatment. Similar to step 160, step 180 can include the use of encrypted authentication codes or by unique, manufacturer supplied identity numbers. As such, step 180 assures that the proper optical fiber 106 is being used with the laser unit 100 for the proper medical procedure. Once authenticated, the medical procedure for the patient can be performed. After the medical procedure is performed, the optical fiber 106 via the smart card system 143 can optionally be de-authenticated by the control system to prevent re-use or in the event that the optical fiber 106 is to be re-used following sterilization and inspection, the smart card can be de-authenticated, and upon completion of the sterilization and inspection procedures, the smart card can be re-authenticated for use. In addition, information about the use of the optical fiber 106, for example, run specific such as total laser power delivered, total procedures performed and the like, can be written to the smart card. The increased memory capacity of the smart card can allow for storage of a wide variety of additional historical information pertaining to operation of optical fiber 106.

While the above-invention has been described in reference to the use of an optical fiber with the GreenLight PVP or HPS laser systems for the treatment of benign prostate hyperplasia (BPH), utilization of laser light on tissue to vaporize or ablate the tissue has many uses including but not limited to treating erectile dysfunction by improving blood flow in the groin area through removal of tissue that is obstructing blood flow; eliminating small/medium uterine fibroids or hemorrhoids; necrosing tissue through a vaginal incision (or perineal for males) to cause scarring in the abdominal area to simulate what mesh does to help cure incontinence or prolapse; removing the outer layer of the uterus to eliminate menorrhagia; vaporizing other tissue masses (e.g. cysts) in the gastrointestinal tract or other parts of the body; conducting internal tubal ligations or tissue scarring to naturally create reversible occlusions in the fallopian tubes or to open the opening to the fallopian tube; and pinpointing and severing the vas deferens to perform minimally invasive male sterilization. The same or similar RFID system of the present invention can be used with each of the above and in any other system where appropriate.

Although specific examples have been illustrated and described herein, it will be appreciated by those of ordinary skill in the art that any arrangement calculated to achieve the same purpose could be substituted for the specific examples shown. This application is intended to cove adaptations or variations of the present subject matter. Therefore, it is intended that the invention be defined by the attached claims and their legal equivalents.

What is claimed:

1. A medical laser system comprising:
a laser unit for use in a specified medical procedure, the laser unit operating at the direction of a control system;
an optical fiber operably coupled to the laser unit, the optical fiber including a tip for positioning proximate targeted tissue; and
a memory device associated with the optical fiber, the memory device adapted to allow the control system to access information embedded within the memory device so as to authenticate the optical fiber as suitable to operate with said laser unit.

2. The laser system of claim 1, wherein the memory device comprises a radio frequency identification device integral to the optical fiber.

3. The laser system of claim 1, wherein the memory device comprises a smart card for interfacing with a smart card reader on the laser unit.

4. The laser system of claim 1, wherein the control system and the memory device share a common authentication key, and wherein the authentication key allows the memory device to generate a unique answer in response to a non-repeated prompt from the control system.

5. The laser system of claim 1, wherein the memory device includes a factory settable memory region including manufacturer settable identity numbers that are proprietary to a supplier of the laser unit.

6. The laser system of claim 1, wherein the memory device communicates operational history information of the optical fiber to the control system.

7. The laser system of claim 1, wherein the control system writes procedure related information to the memory device.

8. The laser system of claim 1, wherein the control system is adapted to de-authenticate the memory device to prevent further use of the optical fiber in the laser unit.

9. The laser system of claim 1, wherein said laser unit is configured to provide photoselective vaporization of the prostate tissue through said optical fiber.

10. A method of authenticating an optical fiber for use with a medical laser system:
providing an optical fiber having a memory device specific to the optical fiber, the memory device including fiber information unique to the optical fiber;
connecting the optical fiber to a laser unit; and
authenticating use of the optical fiber with the laser unit by accessing the fiber information with a control system on the laser unit, whereby the control system verifies compatibility of the optical fiber with the laser unit.

11. The method of claim 10, wherein the memory device comprises a radio frequency identification device.

12. The method of claim 11, further comprising:
embedding the radio frequency identification device within the optical fiber.

13. The method of claim 12, further comprising:
generating a low power radio frequency field in the laser unit to allow the control system to communicate with the embedded radio frequency identification device.

14. The method of claim 10, wherein the memory device comprises a smart card.

15. The method of claim 14, further comprising:
inserting the smart card into a smart card reader on the laser unit such that the control system receives information ready by the smart card reader.

16. The method of claim 10, further comprising:
obtaining a treatment history of the optical fiber with the control system.

17. The method of claim 10, further comprising:
writing procedure related information to the memory device.

18. The method of claim 10, further comprising:
de-authenticating the memory device to prevent further use of the optical fiber in the laser unit.

19. The method of claim 10, wherein authenticating use of the optical fiber with the laser unit by accessing the fiber information with a control system on the laser unit further comprises:
sharing a common authentication key between the control system and memory device such that the memory device generate a unique answer in response to a non-repeated prompt from the control system.

20. The method of claim 10, wherein authenticating use of the optical fiber with the laser unit by accessing the fiber information with a control system on the laser unit further comprises:
creating a factory settable memory region within the memory device including manufacturer settable identity numbers that are proprietary to a supplier of the laser unit.

21. The method of claim 10, wherein the laser unit is configured to provide photoselective vaporization of the prostate tissue through said optical fiber.
